(19) United States
(12) Patent Application Publication
(10) Pub. No.: US 2008/0234565 A1
(43) Pub. Date: Sep. 25, 2008

(54) Ambulatory Physiological Monitor Having A Patient-Activated Emergency Alert Cancellation Feature

(75) Inventors: James Knox Russell, Bainbridge Island, WA (US); Thomas Dean Lyster, Bothell, WA (US)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 10/598,331

(22) PCT Filed: Feb. 1, 2005
(86) PCT No.: PCT/IB05/50429

§ 371 Date: Aug. 24, 2006

Related U.S. Application Data
(60) Provisional application No. 60/548,026, filed on Feb. 26, 2004.

(51) Int. Cl. A61B 5/0408; A61B 5/044

(52) U.S. Cl. 600/382; 600/509

(57) ABSTRACT

An ambulatory physiological monitor (10) is provided. The monitor includes at least one sensor for detecting at least one physiological parameter of a patient and a housing (42) adapted to be secured to the patient. A circuit (50) is located in the housing for receiving and processing a signal representative of the physiological parameter from the sensor to generate recordable physiological data and for determining if the data exceeds a preestablished alarm limit. An event indicator (12) is coupled to the housing for notifying the patient when the alarm limit has been exceeded. A wireless transmitter (28), operationally coupled to the circuit, is located in the housing for transmitting an emergency notification when the alarm limit has been exceeded. A patient-operable actuator (22) is coupled to the housing for preventing transmission of the emergency notification by the wireless transmitter upon activation by the patient within a predetermined time after the alarm limit has been exceeded.

The present invention relates generally to ambulatory physiological monitors, and more particularly to an ambulatory physiological monitor that transmits an alarm or warning in the event of a patient emergency.

Monitoring of human physiological status data has received a high and growing level of interest in a number of medical, industrial, scientific and recreational disciplines. For example, monitoring of electrocardiography (ECG) data is a useful tool in diagnosing the condition of a patient's heart. Conventional physiological monitors allow instantaneous values of the physiological parameters to be viewed.

An ambulatory physiological monitor is a portable electronic device that is secured to a patient for extended periods of time so that the physiological parameter detected by the device can be continuously monitored. The monitors may or may not include a recording unit for storing the data for subsequent analysis and/or a wireless transmitter for transmitting the data to a remote location where it can be analyzed.

Many ambulatory monitors are designed to detect events that only occur infrequently, but are life-threatening when they do occur such as a ventricular arrhythmia, for example. The effectiveness of an ambulatory monitor depends on its acceptance both by the patient and the responding individual or individuals. One factor that may make patients reluctant to wear such monitors is the fear that the monitor will generate a false alarm, possibly resulting in the unnecessary deployment of emergency responders, which can be both expensive and embarrassing to the patient. Likewise, the emergency responders may be unwilling to respond to an automatically generated alarm unless they can be confident that there is in fact an emergency.

Accordingly, it would be desirable to provide an ambulatory physiological monitor that only transmits an emergency notification when an actual emergency situation arises.

In accordance with the present invention, an ambulatory physiological monitor is provided. The monitor includes at least one sensor for detecting at least one physiological parameter of a patient and a housing adapted to be secured to the patient. A circuit is located in the housing for receiving and processing a signal representative of the physiological parameter from the sensor to generate recordable physiological data and for determining if the data exceeds a pre-established alarm limit. An event indicator is coupled to the housing for notifying the patient when the alarm limit has been exceeded. A wireless transmitter, operationally coupled to the circuit, is located in the housing for transmitting an emergency notification when the alarm limit has been exceeded. A patient-operable actuator is coupled to the housing for preventing transmission of the emergency notification by the wireless transmitter upon activation by the patient within a predetermined time after the alarm limit has been exceeded.

In accordance with one aspect of the invention, the event indicator is an audio transducer.

In accordance with another aspect of the invention, the event indicator is a mechanical transducer.

In accordance with another aspect of the invention, the event indicator generates a physical stimulus that increases in intensity over a predetermined period of time after the alarm limit has been exceeded.

In accordance with another aspect of the invention, the patient-operable actuator is a button.

In accordance with another aspect of the invention, the patient-operable actuator is pressure activated.

In accordance with another aspect of the invention, a method is provided for transmitting an emergency notification from an ambulatory monitor upon detection of a physiological parameter of a patient that deviates by a pre-established amount from an acceptable value. The method begins by detecting at least one physiological parameter of the patient. A signal representative of the physiological parameter is received and processed to generate recordable physiological data. If the data is determined to exceed a pre-established alarm limit the patient is notified. An emergency notification is transmitted after the alarm limit has been exceeded for a predetermined period of time unless canceled by the patient within the predetermined period of time.

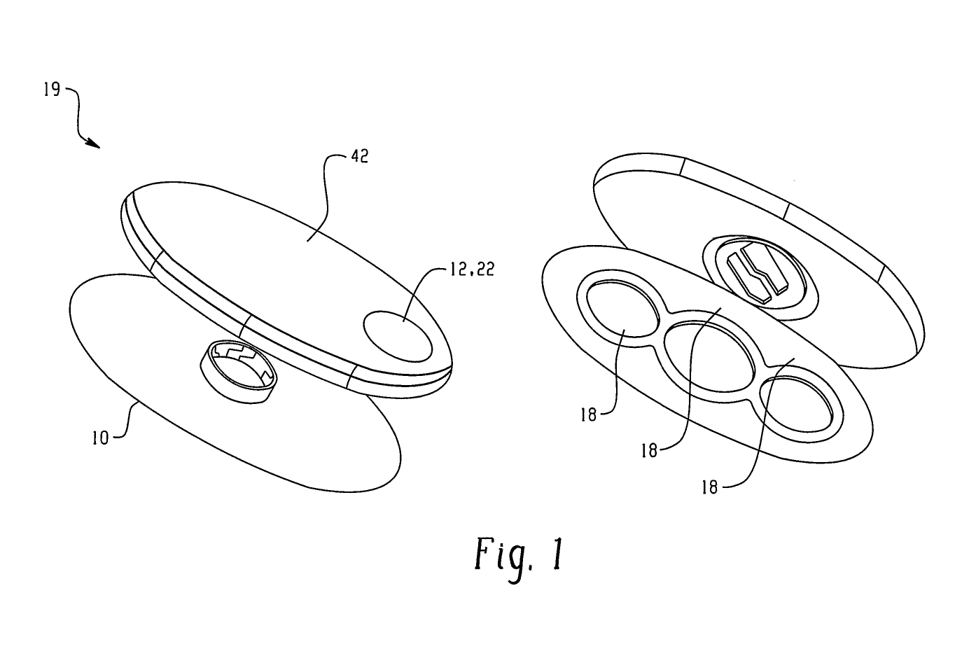

FIG. 1 shows a perspective view of an ambulatory physiological monitor in accordance with the present invention.

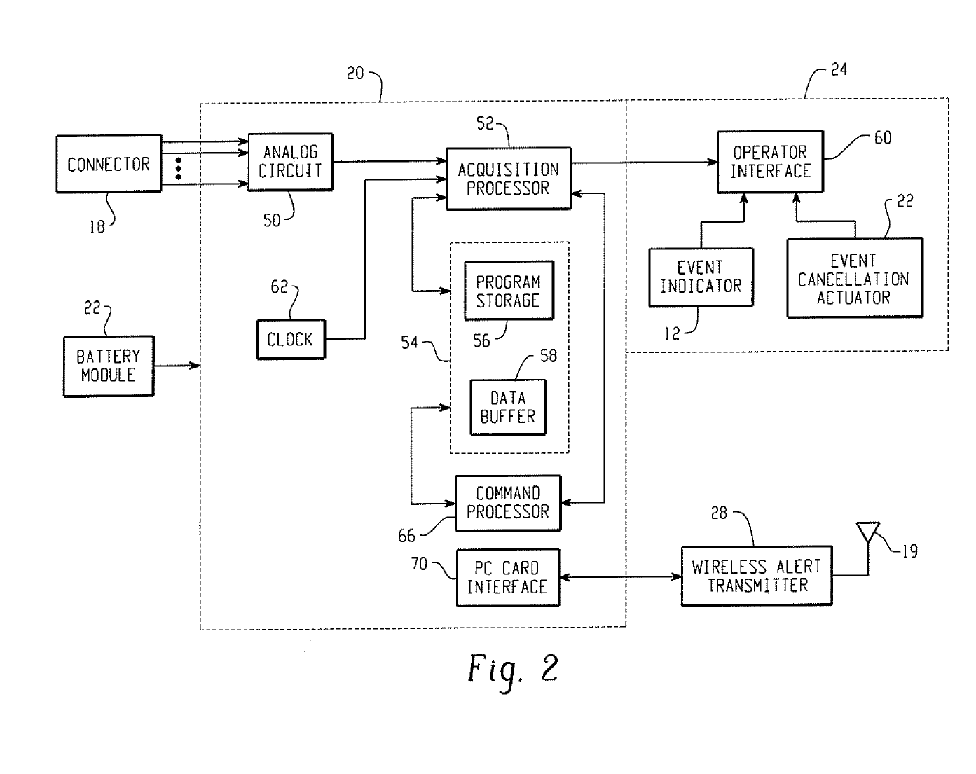

FIG. 2 shows a block diagram of one embodiment of the ambulatory physiological monitor shown in FIG. 1.

The present inventors have recognized that false alarms can be reduced or even eliminated by first notifying the patient with a discreet signal before the alarm is generated. The patient is also provided with an opportunity to cancel the alarm before it occurs. If the patient does not cancel the alarm, presumably because the patient is incapacitated or unconscious, the likelihood is high that a true emergency exists. The discreet signal may escalate in several steps over a limited period of time before the alarm is ultimately generated. In this way the patient is given the maximum opportunity to cancel the alarm.

By signaling the patient with a tiered sequence of signals that are initially discreet but which subsequently become more and more prominent while giving the patient the opportunity to cancel the alarm at any point in the sequence, false alarms are less likely to occur. In this way the patient is assured of discreet notification and thus can avoid any embarrassment and inconvenience that could arise if an emergency condition is falsely reported. At the same time, the absence of alarm cancellation confirms the likelihood that that there is a true emergency, so that once the alarm notifies the responder the likelihood that it represents a false alarm is greatly reduced.

A perspective view of an ambulatory physiological monitor 10 in accordance with the present invention is shown in FIG. 1. The elements of the monitor 10 are enclosed within a housing 42. An event indicator 12, an event cancellation actuator 22, a patient connector 18 and an antenna 19 are incorporated in the monitor 10. The patient connector 18 provides an electrical connection between the circuitry of the ambulatory physiological monitor 10 and sensors or other transducers affixed to the patient for monitoring the patient's condition. The monitor 10 is carried by an ambulatory patient during the patient's normal activities, and the patient's physiological parameters of interest are recorded while the patient is ambulatory.

It should be noted that while monitor 10 is typically designed to be simple and compact, in some embodiments of the invention the monitor may include additional features such as a recording unit for storing the data for subsequent analysis and/or a wireless transmitter for transmitting the data to a remote location where it can be analyzed. However, such features are optional and should not be construed as a limitation on the invention.

The event indicator 21 may be an audio alert or a mechanical vibrator capable of signaling the patient, at least initially, without drawing the attention of bystanders. The event cancellation actuator 22 permits the user, provided he or she is capable of responding and does not desire assistance, to signal the device in order to cancel the generation of alert signals capable of drawing the attention of bystanders or of initiating a chain of events that results in the deployment of emergency medical assistance. The antenna 19 is used for wireless communication as described below.

A block diagram of one embodiment of the ambulatory physiological monitor 10 is shown in FIG. 2. The contacts in patient connector 18 are connected to an analog circuit 50 in electronics module 20. As indicated above, sensors attached to the patient are electrically connected through connector 18 to the monitor 10. The analog circuit 50 amplifies and processes physiological signals from the patient sensors. The outputs of analog circuit 50 are connected to an acquisition processor 52, which controls a portion of the monitor operation and converts amplified analog physiological signals into digital data. The acquisition processor 52 is connected to a memory 54 which includes a program storage area 56 and a data buffer 58. The program storage area 56 is used to store a program for controlling operation of the acquisition processor 52. Data buffer 58 provides temporary storage of physiological data. A real-time clock 62 is connected to acquisition processor 52.

A command processor 66 is connected to acquisition processor 52 and to memory 54. The program storage area 56 is used to store programs for controlling operation of the command processor 66. The command processor 66 also controls transmission and reception of information through wireless alert transmitter 28 as described below. Portions of the monitor 10 may be powered down when not in use to save battery power.

Wireless transmitter 28 sends alerts to a remote location to notify the emergency responder. In some embodiments of the invention the wireless transmitter 28 may send the data to a device that in turn forwards the data to the responder over a telephony or computer network. For example, if the monitor is to be used primarily in the patient's residence, the wireless transmitter 28 may forward the data to a specially enabled telephone that is also located in the residence.

In operation, the acquisition processor 52 acquires signals representing the physiological parameter being measured from analog circuit 50, converts the signals to data and stores the data in data buffer 58. The acquisition processor 52 also checks alarm limits (e.g., a life-threatening event) with respect to the physiological data. For example, if physiological data is being measured, the acquisition processor 52 may check the data for the occurrence of abnormal heartbeats. The alarm limits may be stored in program storage area 56 or any other appropriate location that can be accessed by acquisition processor 52. If the acquisition processor 52 determines that an alarm limit has been exceeded, indicative of an emergency situation, the acquisition processor 52 activates the event indicator 12. The event indicator 12 may be a visual, audio, or any other indicator means for notifying the patient that the alarm limit has been exceeded. For example, the indicator 12 may be an audio transducer that plays a tone or tune. Alternatively, the indicator 12 may be a mechanical transducer that causes a tactile stimulation such as by causing the monitor to vibrate. The event indicator 12 should be able to gradually increase in intensity (e.g., volume, brightness, tactile stimulation) to ensure that the patient has an opportunity to respond to it.

Unless the patient intervenes as described below, after a predetermined period of time has elapsed after the event indicator 12 has been activated, the acquisition processor 52 will cause the wireless transmitter to transmit an emergency notification to summon an emergency responder. In particular, acquisition processor 52 starts the command processor 66. The acquisition processor 52 causes the command processor 66 and the wireless transmitter 28 to be powered up. Then a command is sent to the command processor 66 requesting that an emergency notification be sent. The notification is then transmitted via the wireless transmitter 28.

If the event indicator 12 is activated to alert the patient and the patient recognizes that a false alarm is about to be generated because the alarm limit has been erroneously exceeded, the patient can use the event cancellation actuator 22 to cancel the transmission of the emergency notification. The event cancellation actuator 22, which may be a button, pressure switch or the like, prevents the acquisition processor 52 from powering up the command processor 66, thereby preventing the emergency notification from being sent by the wireless transmitter. If the patient does not respond to the event indicator by use of the event cancellation actuator 22, the event indicator 12 will increase in intensity over a predetermined period of time (e.g., one minute) to ensure that the patient has been alerted. After the predetermined time period has elapsed without activation of the event cancellation actuator 22, the acquisition processor 52 will begin the aforementioned process to send the emergency notification.

Although various embodiments are specifically illustrated and described herein, it will be appreciated that modifications and variations of the present invention are covered by the above teachings and are within the purview of the appended claims without departing from the spirit and intended scope of the invention. For example, while the various components have been depicted as discrete elements, those of ordinary skill in the art will recognize that the functionality of those elements may be embodied in hardware, software, or any combination thereof, and thus are not necessarily embodied in discrete physical components.

1. An ambulatory physiological monitor, comprising:
at least one sensor for detecting at least one physiological parameter of a patient;
a housing adapted to be secured to the patient;
a circuit located in said housing for receiving and processing a signal representative of the physiological parameter from the at least one sensor to generate recordable physiological data and for determining if said data exceeds a pre-established alarm limit;
an event indicator coupled to said housing for notifying the patient when the alarm limit has been exceeded;
a wireless transmitter operationally coupled to the circuit and located in said housing for transmitting an emergency notification when the alarm limit has been exceeded; and
a patient-operable actuator coupled to said housing for preventing transmission of the emergency notification by the wireless transmitter upon activation by the patient within a predetermined time after the alarm limit has been exceeded.

2. The monitor of claim 1 wherein said event indicator is an audio transducer.

3. The monitor of claim 1 wherein said event indicator is a mechanical transducer.

4. The monitor of claim 2 wherein said event indicator generates a physical stimulus that increases in intensity over a predetermined period of time after the alarm limit has been exceeded.

5. The monitor of claim 4 wherein said patient-operable actuator is a button.

6. The monitor of claim 4 wherein said patient-operable actuator is pressure activated.

7. A method of transmitting an emergency notification from an ambulatory monitor upon detection of a physiological parameter of a patient that deviates by a pre-established amount from an acceptable value, said method comprising the steps of:
detecting at least one physiological parameter of the patient;
receiving and processing a signal representative of the physiological parameter to generate recordable physiological data;
determining if said data exceeds a pre-established alarm limit;
notifying the patient when the alarm limit has been exceeded; and
transmitting an emergency notification after the alarm limit has been exceeded for a predetermined period of time unless canceled by the patient within said predetermined period of time.

8. The method of claim 7 wherein the transmitting step is performed with a patient-operable actuator located on the monitor.

9. The method of claim 8 wherein the notification step is performed by an event indicator located on the monitor.

10. The method of claim 9 wherein said event indicator is an audio transducer.

11. The method of claim 8 wherein said event indicator is a mechanical transducer.

12. The method of claim 8 wherein said event indicator is an audio transducer.

13. The method of claim 8 wherein said event indicator generates a physical stimulus that increases in intensity over a predetermined period of time after the alarm limit has been exceeded.

14. The method of claim 7 wherein said patient-operable actuator is a button
